(19) United States
(12) Patent Application Publication
(10) Pub. No.: US 2006/0183166 A1
(43) Pub. Date: Aug. 17, 2006

(54) Arrays of supported biomembranes and uses thereof

(76) Inventors: Michael Mayer, Ann Arbor, MI (US); Sheereen Majd, Ann Arbor, MI (US)

Correspondence Address: HARNESS, DICKEY & PIERCE, P.L.C., P.O. BOX 828, BLOOMFIELD HILLS, MI, 48303, US

(21) Appl. No.: 11/351,496

(22) Filed: Feb. 10, 2006

Related U.S. Application Data
(60) Provisional application No. 60/652,029, filed on Feb. 11, 2005.

(51) Int. Cl. G01N 33/542; G01N 33/53

(52) U.S. Cl. 435/7.9

(57) ABSTRACT

A process for making a biomembrane array comprising providing a first substrate having a discrete zone and a second substrate. A first ink having a proteoliposome is then loaded onto the discrete zone to form a loaded zone. The loaded zone is then contacted to the second substrate such that the first ink is deposited from the loaded zone on the second substrate, thereby forming a biomembrane array on the second substrate.

### CROSS-REFERENCE TO RELATED APPLICATIONS

This application claims the benefit of U.S. Provisional Application No. 60/652,029, filed on Feb. 11, 2005. The disclosure of the above application is incorporated herein by reference.

### FIELD

The present teachings relates to a stamping method for placing biomembranes on a support and methods and uses thereof.

### BACKGROUND

Membrane proteins represent the single most important class of drug targets. Arrays of membrane proteins have been widely used for investigating lipid-protein interaction, protein-protein interactions, and drug-membrane interactions. Considering all these growing applications of arrays of membrane proteins, the ability to pattern arrays of lipid bilayers containing functional membrane proteins has become essential for performing multiplexed, high information content assays. Lipid vesicles are widely used as mimics for cell membranes. Many cellular components including a large number of potential drug targets are associated with cell membranes. Lipid bilayers on solid supports are especially challenging because they are two-dimensional fluids. Methods for patterning and displaying membrane components such as lipids and some membrane tethered or anchored proteins have been described on patterned supported bilayers. Often these methods fail for membrane tethered proteins and they nearly always fail for integral membrane proteins.

This failure is believed to be caused by interactions between regions of the membrane protein that are outside the lipid milieu and therefore interact strongly with the substrate surface. This can cause denaturation of the protein with loss of function or it can limit the lateral mobility of the membrane protein, often important for function. Some investigators have described strategies for lifting the supported bilayer away from the underlying solid support by the use of polymer cushions or by some tethering strategies. In most cases, a substantial immobile fraction of proteins is observed and the activity of the proteins in such cushioned bilayers is often reduced. Existing techniques to fabricate membrane protein arrays have four shortcomings: 1) they employ serial fabrication of spots, a time consuming procedure, 2) this slow fabrication can result in drying, and thus inactivation, of delicate membrane proteins, 3) they consume significant amounts of precious membrane preparations, and 4) they are prone to cross-contamination due to the fluids that are involved.

It has been previously shown that certain formulations of lipid bilayers can be stamped by using microcontact printing. The resultant lipid bilayers did not diffuse beyond 106% of the original printing areas. However, the printing stamp used to print phospholipids could not be successfully applied to stamping biological membrane proteins, because theoretically, the proteins would denature on the stamp without further protection.

### SUMMARY

According to the principles of the present teachings, a process is provided for manufacturing a biomembrane array comprising providing at least one first substrate or stamp having one or more discrete zones wherein each zone having a surface, at least one proteoliposome-containing ink and at least one second substrate having at least one surface. The first substrate is loaded with at least one proteoliposome-containing ink onto the surface of a zone or zones of the first substrate or substrates to form at least one loaded zone. The contacting the surface of one or more loaded zones with a surface of at least one second substrate results in the deposition of at least two spots on said surface of the second substrate, thereby forming a biomembrane array on the second substrate.

The present teachings further provide a method of using a functional membrane protein array to detect membrane-bound biochemical interactions, comprising (i) incubating the array of claim 300 under physiological conditions effective to enable the association of the membrane bound protein and its cognate molecule and, (ii) measuring the quality and quantity of association between the coupled membrane protein and its bound cognate molecule. In some embodiments, the membrane bound protein is selected from the group comprising integral membrane proteins, transport proteins, receptors, enzymes, anchor proteins, heat shock proteins, trafficking proteins, cytokines, voltage and ligand gated ion channels.

In some embodiments, the methods and processes of the present teachings offer several advantages over the prior approaches among others, including, the fragile and easily destroyed biomembranes are stable in hydrogel stamps allowing for multiple copying of biomembrane arrays. The arrays require small volumes of difficult to isolate biological samples. High throughput screening can be performed using arrays made from reproducible stamping techniques that allows the measurement and detection of real time membrane protein interactions with drugs, enzymes and other biological molecules.

Further areas of applicability of the present teachings will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present teachings will become more fully understood from the detailed description and the accompanying drawings, wherein:

FIG. 1 is a series of fabrication steps for making an array in accordance with some embodiments of the present teachings;

FIG. 2 is a series of fabrication steps for making an array in accordance with some embodiments of the present teachings;

FIG. 3 is a schematic cross-sectional view illustrating the biospecific immobilization of proteoliposomes with embedded transmembrane proteins;

FIG. 4(a) is a graph illustrating the flourescence intensity after 6 and 100 stampings of the array using a hydrogel stamp according to the principles of the present teachings;

FIG. 4(b) is a flourescence image from a FRAP experiment performed on the resultant array after 100 stamping events and 8 minutes of photobleaching; and

FIG. 5 is a series of stamping steps according to the principles of the present teachings.

### DETAILED DESCRIPTION

The following description is merely exemplary in nature and is not intended to limit the present disclosure, application, or uses.

Arrays of the Present Teachings

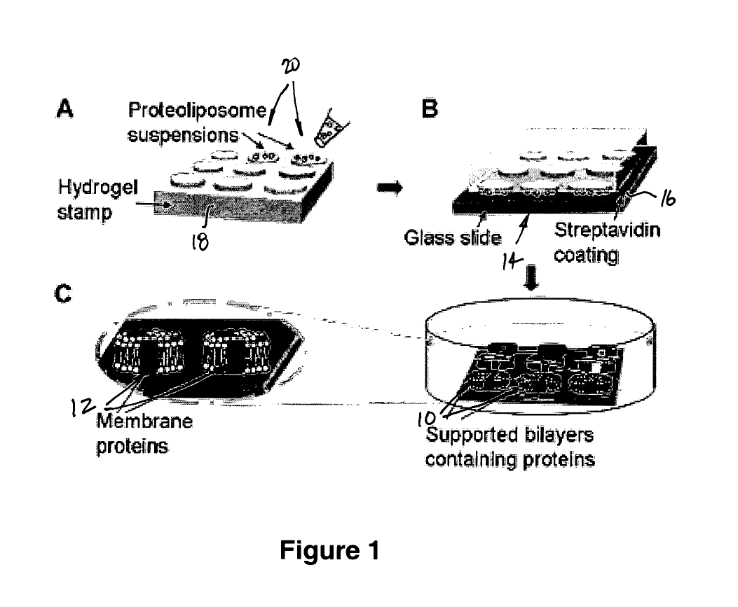

As shown in FIG. 1, the array (10) of the present teachings can include a solid or semi-solid substrate material (14) having an optional surface coating material (16) having a plurality of supported lipid bilayers containing active biomembranes in the form of reaction spots (16) covering the surface of the coated substrate (14). In some embodiments, the biomembranes can comprise stably supported lipid bilayers having biologically active proteins or membrane proteins or other molecules functionally embedded within the lipid bilayers which can interact with other molecules, ligands, proteins, cells, and the like. Each reaction spot (16) on the array (10) comprises a biological membrane of known or unknown composition and, in some embodiments, comprises a membrane bound protein. In some embodiments, array (10) can comprise substrates having predetermined spots of biomembranes or proteoliposomes containing lipid bylayers and membrane proteins functionally embedded within the fluid lipid bilayer. In some embodiments, proteoliposomes can include liposomes that contain membrane proteins that are functionally active as structural proteins. In some embodiments, proteoliposomes also can include receptors, ion channels, docking molecules, integrins, adhesins, enzymes and other proteins capable of interacting with ligands.

In some embodiments, a ligand can be a molecule that is capable of specifically binding to a receptor. Binding of the ligand to the receptor is typically characterized by a high binding affinity, i.e., K (a)>10 (5), and can be detected either as a change in the receptor's function (e.g., the opening of an ion channel associated with or part of the receptor) or as a change in the immediate environment of the receptor (e.g., detection of binding by surface plasmon resonance). Ligands for incorporation into expanses of lipids in vitro (e.g., supported bilayers) may be either purified from cells, recombinantly expressed, or, in the case of small ligands, chemically synthesized. It should be understood that in some embodiments, binding is specific if it results from a molecular interaction between a binding site on a receptor and a ligand rather than from non-specific sticking of a ligand to a receptor. In cases where the ligand binds the receptor in a reversible manner, specificity of binding can be confirmed by competing off labeled ligand with an excess of unlabeled ligand according to known methods. Non-specific interactions can be minimized by including an excess of a non-specific protein (e.g., BSA) that does not have binding sites for either the ligand or receptor.

Reaction spot (16) may comprise the same or multiple different membrane proteins. For example, two or more different proteins involved in a heterodimer pair can be included in one spot. The spots on the array may be any convenient shape, but will typically be determined by the volume and deposition technique of the agarose or hydrogel stamp. The spot can contain a sub-microliter solution of proteoliposome or lipid bilayers containing a single or mixture of proteins embedded within the lipid bilayer. The proteoliposomes diffuse from the agarose or hydrogel inked stamp and imprint structurally supported lipid bilayers in various shapes including, circular, elliptoid, oval, annular, or some other analogously curved shape, generally depending on the ink volume and type of hydrogel stamp used. The density of the all of the spots on the surface of the substrate, i.e. can be at least about 1/cm² and usually at least about 16/cm² but does not exceed about 1000/cm², not to exceed about 600 cm², not exceed about 256/cm², does not exceed about 200/cm², and in some embodiments usually does not exceed about 64/cm². In some embodiments, automated robotic procedures could increase the density of spots imprinted if the stamp used to imprint the lipid bi-layers could be manufactured using miniturization techniques. The spots may be arranged in any pattern across or over the surface of the array, generally dependent on the shape of the imprinting stamp, which can be molded to conform any given shape. In some embodiments, the pattern of spots will be imprinted typically in the form of a grid across the surface of the coated substrate.

In the arrays of the present teachings, the spots are stably associated with the surface of a coated or non-coated substrate. By “stably associated” it is meant that the spots maintain their position relative to the substrate under experimental conditions such as binding and/or washing conditions. As such, the biomembranes which make up the spots can be non-covalently or covalently stably associated with the substrate surface. Examples of non-covalent association include non-specific adsorption, binding based on electrostatic (e.g. ion, ion pair interactions), hydrophobic interactions, hydrogen bonding interactions, surface hydration force and the like, and specific binding based on the specific interaction of an immobilized binding partner and a membrane bound protein. In some embodiments, the phospholipids used to synthesize the lipid bilayers are specifically conjugated with molecules of biotin. When the lipid bilayers are labelled with biotin, the substrate can be coated with streptavidin. The binding between the substrate and the biomembrane is structurally enhanced and subsequent manipulations can be performed due to the increased bond attraction between biomembrane, including attraction between the lipids in the biomembrane and the substrate and between the membrane protein and the substrate. Specific binding-induced immobilization includes, for example, antibody-antigen interaction, generic ligand-receptor binding, lectin-sugar moiety interaction, etc. Examples of covalent binding include covalent bonds formed between the spot biological membranes and a functional group present on the surface of the substrate, e.g. —NH₂, where the functional group may be naturally occurring or present as a member of an introduced coating material. In some embodiments coating materials can also include one or more of the following, streptavidin, positively charged amino groups, wheat germ agglutinin, collagen, lysine, albumin and any natural or recombinant antibody which binds to a membrane protein and positively charged polymers polyvinylamines, polyallylamines, polyethyleneimines and modified polyethyleneimines.

In some embodiments, the array comprises substantially identical spots (e.g., spots comprising the same proteins) or a series of substantially identical spots that are reacted with a different analyte (target) when the array is being screened. Spots that are printed identically can in some embodiments refer to examples when the hydrogel stamp is inked with the one set of proteoliposomes containing one or more different membrane proteins and the stamp is used to deposit the same composition of proteoliposomes. In some embodiments, substantially identical substrates are arrays on two or more substrates imprinted using the same stamp containing the same composition of proteoliposomes in the same configuration on the stamp.

In some embodiments of the array, the protein included in the spot differs from the protein included on a second spot of the same array. In such an embodiment, a plurality of different proteins are present on separate spots of the array. Typically the array comprises at least about two different proteins. Preferably, the array comprises at least about 10 different proteins. In some embodiments, the array comprises at least about 50 different proteins, comprises at least about 100 different proteins, comprise more than about 1000 different proteins, comprise more than about 10,000 different proteins. In some embodiments the array may even optionally comprise more than about 10⁵ to about 10⁸ different proteins.

For example, an array of the invention can include a category of spots, each spot containing a different membrane bound protein pertaining to a specific category of proteins or receptors, wherein each category of arrays are repeated several times (5-50 times or more) as part of a larger array or group of arrays. In some embodiments, the receptor can comprise a macromolecule capable of specifically interacting with a ligand molecule. In cells, receptors can be associated with lipid bilayer membranes, such as the extracellular, Golgi or nuclear membranes. Receptors for incorporation into expanses of lipids in vitro (e.g., supported bilayers) can be either purified from cells, recombinantly expressed, or, in the case of small receptors, chemically synthesized.

In some embodiments one or more different target membrane proteins can be screened to determined specific binding with various drugs. In some embodiments, arrays may comprise identical supported biomembranes wherein each protein within the array is the same. In some embodiments the arrays contain a potassium channel ion capable of conducting potassium ions after a current is applied. Screens can be developed in some embodiments to determine whether a known or unknown drug can act as an antagonist or agonist to study drug-ion-channel membrane interactions. The use of a biocompatible stamp for imprinting sensitive biological membranes would enable the creation of arrays having a fixed volume and constant chemical composition of biomembranes, ensuring reproducibility and compatibility in automated testing for example in high throughput parallel screening.

Preparation of the Arrays

Considering the application of membrane arrays for screening protein-membrane or drug-membrane interactions, in some embodiments according to the present teachings, the fabrication method would rapidly create many functional copies of an array of identical, similar or different bilayers while consuming minimal amounts of lipids. In some embodiments, arrays of the present teachings can be created by contact printing of the hydrogel stamp onto the surface of a substrate or a plurality of substrates, which has the ability to produce many copies of arrays of membrane proteins in parallel. In some embodiments. Other array manufacturing procedures require the posts of the stamp to be inked individually, especially when different compositions are to be imprinted. Such an inking procedure can be time consuming and introduce heterogeneity in the stamped arrays. It would therefore be advantageous if a biocompatible stamp—once inked—would store the inking solution and allow multiple transfers without the need for re-inking.

In some embodiments, biomembrane arrays are manufactured using biocompatible microstamping applications with hydrogel stamps allowing for multiple stamping of spots while using minute amounts of isolated biological material. In some embodiments, stamps are fabricated from hydrogels. In some embodiments, the hydrogel can be agarose. In some embodiments, the hydrogel can comprise macromolecular networks swollen in water or biological fluids producing cross-linked polymeric structures by the simple reaction of one or more monomers.

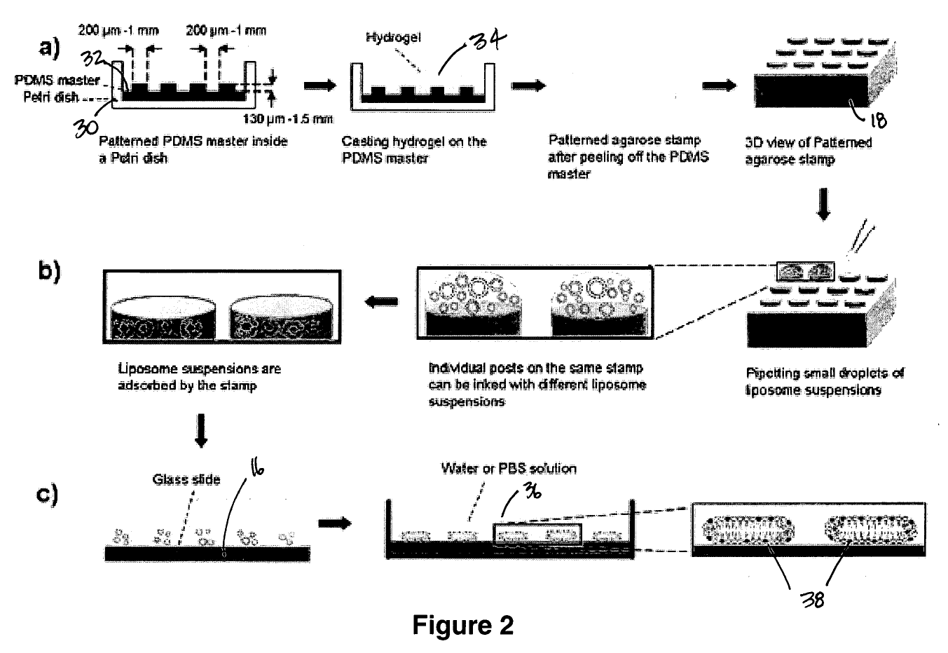

As shown in FIG. 2, a-c, in some embodiments, microcontact stamps are manufactured by casting a hydrogel including, but not limited to, agarose gel onto a patterned PDMS master and peeling off the PDMS master from the agarose gel resulting in a topographically patterned agarose stamp. In some embodiments, the hydrogel to be used in the manufacturing of the stamp can include any biocompatible hydrogel that can be molded into a durable shape, and which is compatible with proteins, lipids, glycolipids, nucleic acids, glycoproteins etc. In some embodiments, the stamp can be manufactured from one or more of the following: agarose, polyacrylamide, collagen, gelatin, alginate, chitosan, pluronic or combinations thereof.

In some embodiments, the agarose stamp is removed from the mold and inverted so that the liposome suspension containing the lipid bilayers and embedded proteins can be transferred to the posts or discrete zones of the stamp. FIG. 1 (a) and FIG. 2(b). In some embodiments a liposome can refer to a roughly spherical, free-standing bilayer consisting of lipids or lipid-related materials. In some embodiments, a liposome can be unilamellar if it contains a single bilayer or multilammellar if it contains several bilayers. A liposome can be a closed surface so that the vesicle contents and molecules outside the vesicle exchange slowly under ordinary conditions. Liposomes can be prepared by sonication of dispersions of lipid components in water or buffer or by extrusion of such solutions through membranes with defined pore sizes. Liposomes can in some embodiments be prepared with diameters from tens of nm to tens of mm. In addition to lipid content, the liposome bilayer can contain proteins, glycoproteins, glycolipids and other biological molecules that are typically associated with biological membranes. Because the environment is like that in a normal cell membrane, proteins are typically fully functional. The inside of the liposome can be used to trap molecules providing a probe for the integrity of the liposome structure, as sensors for changes in properties of the interior (e.g. pH, ion concentrations and the like) or for studies of content mixing upon liposome fusion or rupture. An exemplary liposome is shown in FIG. 2.

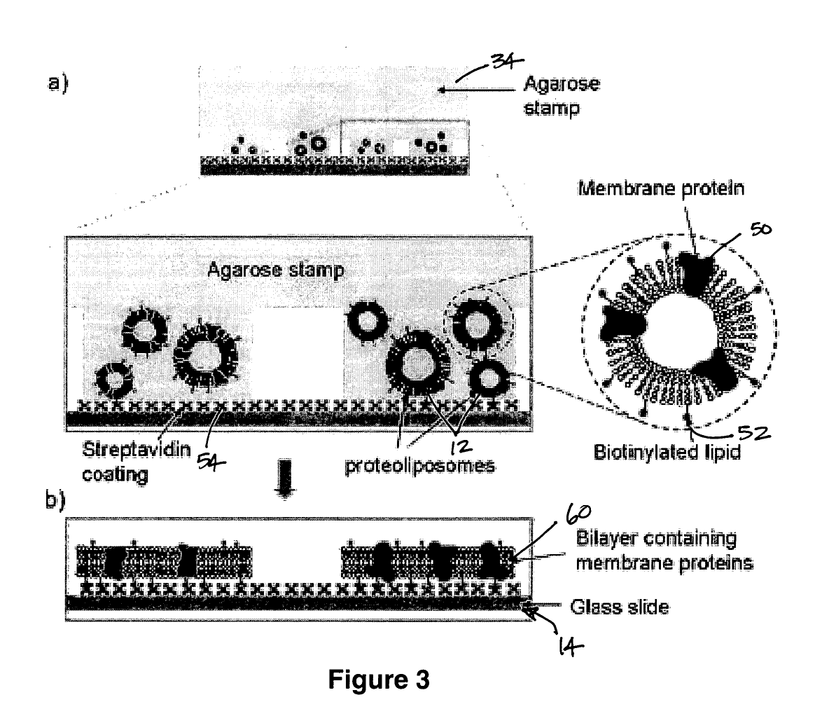

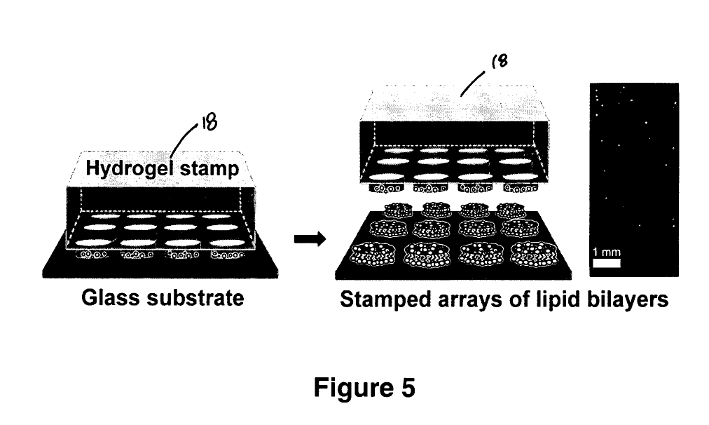

As used herein a “stamp” or “first substrate” is a hydrogel having one or more discrete zones: in some embodiments where the stamp has only one discrete zone, that zone can be the entirety of a surface of the stamp or a portion of that stamp. A zone is referred to as the discrete location where the transfer of proteoliposomes is made. In some embodiments, a stamp can be assembled from multiple substrates, such as multiple blocks or other forms of hydrogel or other material suitable for stamping the ink. In some embodiments of such a stamp, the individual blocks or forms can be arrange such that their stamping surfaces are at least substantially coplanar. Multiple (i.e. at least two) zones can be formed on a first substrate in any method known, for example, two or more different areas of the same surface can be loaded with an ink according to the present teachings, or two or more posts, each having surfaces, raised from the body of the stamp, that are non-contiguous with the raised surface of another post of the stamp. In some embodiments the surface of a post to which a solution of proteoliposomes is added can also be referred to as a zone. Such posts can be molded as part of a single stamp during, e.g., a pouring or casting process, or can be created by stamping, carving, or any other process. In some embodiments, to ink the agarose stamps with 1 mm to 100 μm diameter posts, the agarose stamps can be turned upside down in a Petri dish containing a solution of 0.15 M KCl, such that ˜¾ of the thickness of the stamp is immersed in the KCl solution and the posts (which are facing upwards) are out of the KCl solution. The posts can be inked individually by applying ˜0.2 μL of liposome suspension on top of each post. In some embodiments, the application of the liposome suspension to the hydrogel can be accomplished by any known method in the art of fluidics and liquid manipulation. In some embodiments, the solution of proteoliposomes can be applied manually. In some embodiments, a mechanical or automated fluidics device can apply any volume ranging from sub-milliliter to sub-microliter volume solutions of proteoliposomes onto the posts of the stamp to be patterned. Neighboring posts on the same stamp could be inked with the same or different liposome suspensions. Once the proteoliposome solution is adsorbed by the hydrogel (typically after ˜4 minutes), additional droplets of liposome solution can be applied on top of each post and this process can be repeated as required. In some embodiments, the application of the proteoliposomes can be repeated 2-10 times. In case of stamps with smaller posts (200 μm in diameter), agarose stamps can be prepared by immersing the posts in a solution of proteoliposomes for ˜30 min. After inking the stamps can be turned upside down (200 μm posts facing upwards) and after the stamp has adsorbed all solution, the stamp can be used for stamping onto a planar substrate in some embodiments a glass slide, as shown in FIG. 3 and FIG. 5. In the beginning of a stamping series, the inked stamps can stamped 4-7 times on clean glass slides to remove excess solution of proteoliposomes from the stamp.

To form arrays of lipid bilayers, the inked hydrogel stamp can be placed in contact with clean coated or non-coated substrate for 5-10 seconds. In some embodiments, the substrate is a clean glass slide. After removing the stamp from the substrate, the substrate containing the supported biomembranes were immediately immersed in water or PBS solution as shown in FIG. 2(c). In some embodiments the stamping procedures can be carried out at room temperature with at least 55% humidity. When the arrays are prepared in environments less than <50% humidity the resulting supported biomembranes contain lipid bilayers with reduced fluidity. In some embodiments, the arrays can be prepared in environments with less than 50% humidity and still retain proper fluidity if an aliquot of glycerol is added to the proteoliposomes. Typically, the stamped spots of lipid bilayers can retain their fluidity even after storing them for two weeks in buffer solution.

As shown in FIG. 2, the molded stamp 18 is then inked manually with the biomembrane solution 24 on the top of the post 22. The solution typically forms a meniscus and gradually diffuses into the hydrogel as shown in 26. In some embodiments small droplets (˜0.2 μL) of liposome suspension can be added on top of each post. In some embodiments, 0.1 nL of liposome suspension can be added, 0.5 nL of liposome suspension can be added, 1 nL of liposome suspension can be added, 50 nL of liposome suspension can be added, 0.1 μL of liposome suspension can be added, 0.2 μL of liposome suspension can be added, 05. μL can be added, 1 μL of liposome suspension can be added. In some embodiments, the hydrogel stamp posts can be inked at least up to ten times with various volumes of liposome suspension as described above. Nanometer sized proteoliposomes (10-150 nm) inside the droplet diffuse into the hydrogel and the liposome suspension can be absorbed readily by the hydrogel.

Without being limited to theory, supported lipid bilayer or biomembrane spots can be formed by diffusion of proteoliposomes through the gel and subsequent spreading of these proteoliposomes onto the substrate at the areas of contact between the stamp and the substrate forming functional biomembranes. In some embodiments, greater stability and support of the fragile biomembranes can be achieved when the inked hydrogel is stamped onto a substrate coated with a biological material. As defined herein, a biological coating material can be any material coating that binds to the biomembrane in a non-covalently or covalently stably associated manner with the substrate surface. Examples of non-covalent association include non-specific adsorption, binding based on electrostatic (e.g. ion, ion pair interactions), hydrophobic interactions, hydrogen bonding interactions, surface hydration force and the like, and specific binding based on the specific interaction of an immobilized binding partner and a membrane bound protein. Specific binding-induced immobilization includes, for example, antibody-antigen interaction, generic ligand-receptor binding, lectin-sugar moiety interaction, etc. In some embodiments the substrate can be a clean glass slide coated with a solution of strepavidin.

In some embodiments, stamped biomembrane arrays on glass slides can be immersed in water or PBS buffer immediately after removal of the stamp from the substrate. In some embodiments, the biomembrane arrays were then ready for inspection, binding assays, or storage. In some embodiments, stamps can be manufactured from 4% agarose gel, which has a pore size that is sufficiently large to allow for the diffusion of macromolecules and small liposomes ranging from 10-80 nm (the pore size of 2% agarose gel is ˜470 nm). In some embodiments, this capability makes it possible to store inking solution in the stamp while replenishing molecules at the surface of the stamp and thus to perform multiple stamping of biomolecules, without the need to reink the stamp.

Substrate

In some embodiments, the substrate can comprise of any smooth surfaced material including, but not limited to glass, plastics and metals. The substrates of the subject arrays comprise at least one surface on which the pattern of probe spots is present, where the surface may be smooth or substantially planar, or have irregularities, such as depressions or elevations. The surface on which the pattern of spots is present may be modified with one or more different layers of coating materials that serve to modify the properties of the surface in a desirable manner and will be discussed in more detail below. The surface may also be porous.

The substrate can comprise a ceramic substance, a glass, a metal, a crystalline material, a plastic, a polymer or co-polymer, any combinations thereof, or a coating of one material on another. Such substrates include for example, but are not limited to, (semi) noble metals such as gold or silver; glass materials such as soda-lime glass, pyrex glass, vycor glass, quartz glass; metallic or non-metallic oxides; silicon, monoammonium phosphate, and other such crystalline materials; transition metals; plastics or polymers, including dendritic polymers, such as poly(vinyl chloride), poly(vinyl alcohol), poly(methyl methacrylate), poly(vinyl acetate-maleic anhydride), poly(dimethylsiloxane) monomethacrylate, polystyrenes, polypropylene, polyethyleneimine; copolymers such as poly(vinyl acetate-co-maleic anhydride), poly(styrene-co-maleic anhydride), poly(ethylene-co-acrylic acid) or derivatives of these or the like.

The substrate may take a variety of configurations ranging from simple to complex, depending on the intended use of the array. Thus, the substrate could have an overall slide or plate configuration, such as a rectangular or disc configuration. The determination of the substrate material will depend on the detection assays to be performed on the arrays present on the surface of the substrate. For example, if fluorescence is to be used to detect specific interactions between biomembrane proteins present in the array and fluorescently labeled ligands, then a clear plastic or glass substrate could be used to facilitate transmittance of the laser light required for analysis.

The substrate can generally have a rectangular cross-sectional shape, having a length of from about 10 mm to 200 mm, usually from about 40 to 150 mm and more usually from about 75 to 125 mm and a width of from about 10 mm to 200 mm, usually from about 20 mm to 120 mm and more usually from about 25 to 80 mm, and a thickness of from about 0.01 mm to 5.0 mm, usually from about 0.1 mm to 2 mm and more usually from about 0.2 to 1 mm. Other substrate shapes can be employed depending on the down-stream assay detection protocols and in some embodiments the shape of the imprinting stamp.

In some embodiments, coated and non-coated glass slides can be used. Uncoated glass slides (Microslides, No. 2974, Corning, N.Y.) can be cleaned with fresh piranha solution (mixture of concentrated sulfuric acid and 30% hydrogen peroxide) followed by washing with deionized water at least eight times and drying at 180° C. for 2 hours prior to either coating with a coating material or stamping with proteoliposomes.

Biomembranes

In some embodiments of the present teachings, a “biomembrane” as referred to in the present teachings comprises a membrane which may be synthetic or naturally occurring, for example, but not limited to, vesicles, liposomes, monolayer lipid membranes, bilayer-lipid membranes, membranes incorporated with receptors, whole or part of cell membranes, or proteoliposomes (which are referred to herein as liposomes containing membrane proteins), or detergent micelles containing re-folded proteins, or the like. Membranes suitable for use with the present teachings are amphiphilic molecules, for example, but not limited to, phospholipids, sphingomyelins, cholesterol or their derivatives. In some embodiments, the proteoliposomes contained within the stamp are released onto a substrate and upon placement on the substrate become functional biomembranes.

In some embodiments lipid mixtures used to prepare liposomes can include: 99% L-α-phosphatidylcholine form chicken and 1% (w/w) 1,2-dipalmitoyl-sn-glycero-3-phosphoethanolamine-N-(lissamine-rhodamine B sulfonyl) (rh-PE); 97% egg PC and 3% NBD-labelled PE (NBD-PE); mixtures of egg PC/rh-PE or NBD-PE/1,2-dioleoyl-sn-glycero-3-[phospho-L-serine] (DOPS), and mixtures of egg PC/rh-PE/cholesterol. The respective mixing ratios can be varied. Small unilamellar liposomes were produced by tip sonication of 1 mg lipid in 500 μL of an aqueous solution containing 0.15 M KCl, for 2-6 minutes. Before sonication, the lipids can be dissolved in chloroform and 100 μL of a 10 mg/mL lipid solution in chloroform was used to deposit a lipid film on the wall of a 5 mL round bottom flask using a rotatory evaporator under vacuum (starting form −300 torr and going up to −740 torr). Residual traces of chloroform were removed by desiccation under vacuum (˜−740 torr) for at least 1 hour. The prepared liposomes can then be added to prepared membrane proteins for incorporation into the proteoliposomes of the present teachings.

The fluidity of the biomembrane stamped onto substrates thus creating defined supported arrays can be examined by atomic force microscopy experiments. In some embodiments, some of the biomembranes synthesized according to the present teachings revealed a smooth surface of bilayer.

In some embodiments, the biomembrane includes a membrane-protein. Such membrane proteins include, for example, integral membrane proteins, peripheral membrane proteins and receptors (e.g., G protein-coupled receptors, ion-channel proteins, tyrosine kinase-linked receptors, receptor tyrosine kinases, cytokine receptors, and receptors with intrinsic enzymatic activity). In some embodiments, the membrane may be bilayer-lipid membranes incorporated with, but not limited to, ionophores (for example, but not limited to, valinomycin, nonactin, methyl monesin, coronands, cryptands or their derivatives), ion-channels (for example, but not limited to, potassium voltage gated ion channels, etc.) or synthetic or naturally occurring analytes, for example, but not limited to, antibody, enzyme, lectin, dye, chelating agent and the like.

In some embodiments, voltage gated ion channels can comprise Na⁺, K⁺, Ca²⁺, or C₋ ion channels which are membrane-spanning proteins that selectively conduct Na⁺, K⁺, Ca²⁺, Cl⁻ ions across the cell membrane along its electrochemical gradient at a rate of 10⁶ to 10⁸ ions/s. In some embodiments, K⁺ channels can be endowed with a set of salient features: 1) a water-filled permeation pathway (pore) that allows K⁺ ions to flow across the cell membrane; 2) a selectivity filter that specifies K⁺ as permeant ion species; and 3) a gating mechanism that serves to switch between open and closed channel conformations. Since the first gene encoding a K⁺ channel was cloned from Drosophila Shaker mutant more than 200 genes encoding a variety of K⁺ channels have been identified, all containing a homologous pore segment (S5-S6 linker) selective for K⁺ ions. Accordingly, a general classification of K⁺ channels into families is based upon the primary amino acid sequence of the pore-containing subunit. Three groups with six, four, or two putative transmembrane segments are recognized. These include 1) voltage-gated K⁺ channels (Shaker-like) containing six transmembrane regions (S1-S6) with a single pore; 2) inward rectifier K⁺ channels containing only two transmembrane regions and a single pore; and 3) two-pore K⁺ channels containing four transmembranes with two pore regions.

Proteins

The proteins incorporated on the array may be produced by any of the variety of means known to those of ordinary skill in the art. Any type of protein can be incorporated into the lipid bilayers of the present teachings. Typically proteins to be incorporated into lipid-bilayers can remain functional in lipid bilayers. In some embodiments, proteins to be inserted into Proteins added to the liposomes during synthesis of the proteoliposomes are supported and when mixed into the appropriate lipid composition are free to diffuse within the plane of the membrane mimicking a property of cellular membranes that are essential for many cellular function. The number and types of proteins that can be incorporated into the liposomes of the present teachings can include: natural and synthetic polypeptides, dimmers, heterodimers, receptors (including but not limited to aderenergic receptor, angiotensin receptor, cholecystokinin receptor, muscarinic acetylcholine receptor, neurotensin receptor, galanin receptor, dopamine receptor, opioid receptor, erotonin receptor, somatostatin receptor, etc), G proteins, integrins, cytokines, heat shock proteins, trafficking proteins integral structure proteins growth factors, hormones, enzymes gap-junctions, ion channels (including, but not limited to, Sodium, chloride and potassium ion channels comprising:

ATP regulated rKir1.1 (ROMK1) (rat) U.S. Pat. No. 5,356,775,hKir1 1 (ROMIK1)(human) U.S. Pat. No. 5,882,87, Kir1.2, Kir1.3

II. beta.-cell U.S. Pat. No. 5,744,594

III, h.beta.IR U.S. Pat. No. 5,917,02,

IV. HuK.sub.ATP-1 EP 0 768 379 A1

Constitutively Active Kir2.1 (IRK1) U.S. Pat. No. 5,492,825, U.S. Pat. No. 5,670,335, Kir2.2, Kir2.3 G-protein Regulated Kir3.1 (GIK1, KGA) U.S. Pat. No. 5,728,535, Kir3.2 U.S. Pat. No. 5,734,021, Kir3.3 U.S. Pat. No. 5,744,324 Kir3.4 (CIR) U.S. Pat. No. 5,747,278, Kir4.1 (BIR10) Kir5.1 (BIR9), Kir6.1 Kir6.2, Kir7.1 EP 0 922 763 A1

Voltage Regulated

KCNA1 hKv1.1 (RCK1, RBK1, MBK1, MK1, LO2750HuK1)

KCNA2 Kv1.2 (RBK2, RBK5, NGK1, HuKIV)

KCNA3 Kv1.3 (KV3, RGK5, HuKIII, HPCN3,

KCNA4 Kv1.4 (RCK4, RHK1, HuKII)

KCNA5 Kv1.5 (KV1, HPCN1, HK2)

KCNA6 Kv1.6 (KV2, RCK2, HBK2)

KCNA7 Kv1.7 (MK6, RK6, HaK6) U.S. Pat. No. 5,559,009 K.nu.2 (Shab)

KCNB1 Kv2.1 (DRK1, mShab) M64228

KCNB2 Kv2.2 (CDRK1) K channel 2 U.S. Pat. No. 5,710,019

K.nu.3 (Shaw)

KCNC1 Kv3.1 (NGK2)

KCNC2 Kv3.2 (RKShIIIA)

KCNC3 Kv3.3 (KShIIID)

KCNC4 Kv3.4 (Raw3), K.nu.4 (ShaI)

KCND1 Kv4.1 (mShaI, KShIVA)

KCND2 Kv4.2 (RK5, Rat ShaI 1)

KCND3 Kv4.3 (KShIVB)

hKv5.1 (1K8) WO 99/4137 Kv6.1 (K13) Kv7, Kv8.1, Kv9, Delayed Rectifier KvLQT1 U.S. Pat. No. 5,599,673, HERG (erg) PCT WO 99/20760

Calcium regulated

Ca.sup.2+Regulated Big BKCa (hSLO), HBKb3 (.beta.-subunit) PCT WO 99/42575, Maxi-K U.S. Pat. No. 5,776,734, U.S. Pat. No. 5,637,470 Ca.sup.2+ Regulated small

KCNN1 SKCa1

KCNN2 SKCa2

KCNN3 SKCa3

KCNN4 SKCa4 (IKCa1)

GPI-anchored proteins and combinations thereof, even including bacteria and eukaryotic cells.

Preparation for incorporation in the proteoliposomes of the present teachings, the protein or membrane protein can be obtained from cell membranes or optionally be obtained recombinantly. In some embodiments, the membrane protein can be overexpressed using recombinant DNA methods. Expression vectors compatible with bacterial, yeast, insect and eukaryotic cells, preferably those compatible with vertebrate cells, can also be used to form a recombinant DNA molecule that contains a coding sequence. Eukaryotic cell expression vectors are well known in the art and are available from several commercial sources. Typically, such vectors are provided containing convenient restriction sites for insertion of the desired DNA segment. Eukaryotic cell expression vectors used to construct the recombinant DNA molecules can further include a selectable marker that is effective in a eukaryotic cell, preferably a drug resistance selection marker. A preferred drug resistance marker is the gene whose expression results in neomycin resistance, i.e., the neomycin phosphotransferase (neo) gene. Southern et al., (1982) J. Mol. Anal. Genet. 1, 327-341. Alternatively, the selectable marker can be present on a separate plasmid, the two vectors introduced by co-transfection of the host cell, and transfectants selected by culturing in the appropriate drug for the selectable marker.

Transformed Host Cells

The present teachings further provides host cells transformed with a nucleic acid molecule that encodes a membrane protein. The host cell can be either prokaryotic or eukaryotic, including bacterial cells, yeast cells, plant cells, insect cells and animal cells. Eukaryotic cells useful for expression of a miniature protein of the invention are not limited, so long as the cell line is compatible with cell culture methods and compatible with the propagation of the expression vector and expression of the gene product in general. Transformation of appropriate cell hosts with a recombinant DNA molecule encoding a miniature protein of the present teachings is accomplished by well known methods that typically depend on the type of vector used and host system employed. With regard to transformation of prokaryotic host cells, electroporation and salt treatment methods can be employed (see, for example, Sambrook et al., (1989) Molecular Cloning—A Laboratory Manual, Cold Spring Harbor Laboratory Press; Cohen et al., (1972) Proc. Natl. Acad. Sci. USA 69, 2110-2114). With regard to transformation of vertebrate cells with vectors containing recombinant DNA, electroporation, cationic lipid or salt treatment methods can be employed (see, for example, Graham et al., (1973) Virology 52, 456-467; Wigler et al., (1979) Proc. Natl. Acad. Sci. USA 76, 1373-1376).

Successfully transformed cells (cells that contain a recombinant DNA molecule encoding a protein or membrane protein), can be identified by well known techniques including the selection for a selectable marker. For example, cells resulting from the introduction of a recombinant DNA of the present teachings can be cloned to produce single colonies. Cells from those colonies can be harvested, lysed and their DNA content examined for the presence of the recombinant DNA using a method such as that described by Southern, (1975) J. Mol. Biol. 98, 503-517 or the proteins produced from the cell assayed via an immunological method.

Production of Recombinant Membrane Proteins

The present teachings further provides methods for producing a membrane protein of the present teachings using nucleic acid molecules herein described. In general terms, the production of a recombinant form of a protein typically involves the following steps: a nucleic acid molecule is obtained that encodes a protein of the invention, such as the nucleic acid molecule encoding any membrane protein to be incorporated into a liposome for functional membrane studies with drugs and other proteins in an array. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant miniature protein. Optionally the recombinant membrane protein cab be isolated from the medium or from the cells; recovery and purification of the protein may not be necessary in some instances where some impurities may be tolerated. In various embodiments, the membrane proteins are extracted from the host cells by preparing whole cell membrane preparations. These membrane preparations in some embodiments can be added directly to the stamps themselves, as they can function as a membrane bilayer, or the extracted membrane proteins can be admixed with various lipids to form liposomes embedded with membrane proteins.

Membrane proteins can include, for example, GPCRs (for example (nicotinic acetylcholine receptor, sodium and potassium ion channels, etc), receptor tyrosine kinases (e.g. epidermal growth factor (EGF) receptor), and other membrane-bound proteins. Mutants or modifications of such proteins may also be used. Additionally, the membrane proteins can also (or independently) be modified to include an agonist (or peptide) attached at the N-terminus. GPCRs modified in such a way can be constitutively activated (Nielsen, S. M. et al., (2000) Proc. Natl. Acad. Sci. USA, 97: 10277-10281).

Moreover, for GPCR arrays, it is preferable, in some embodiments, that the receptors be immobilized in an oriented manner. For example, to improve performance of GPCR arrays for ligand screening, the GPCRs are oriented with their ligand-binding sites (extracellular domains) to the solution and intracellular domain facing the substrate. This can be accomplished by a number of methods. For example, the surface of the substrate is modified to contain nitrilotriacetic acid (NTA) groups or ethylenediamine triacetic acid (EDTA) groups chelated to nickel. This surface can be used for immobilizing recombinant GPCRs with histidine tags at their C-terminus. Surfaces presenting NTA groups or EDTA groups can be conveniently obtained by silane chemistry on glass or metal oxide surfaces, or via thiol chemistry on gold-coated surfaces. Compounds for these surface chemistries are commercially available (e.g. N-[(3-trimethoxysilyl)propyl)propyl] ethylenediamine triacetic acid;.).

In some embodiments, when the biomembrane spot comprises a single membrane bound protein, only one type of protein is included in each spot of the array. However, in certain situations more than one type of protein is included in each spot. For example, some GPCRs heterodimerize for their biological functions. (Angers, S. et al., Proc. Natl. Acad. Sci. USA, 2000, 97, 3684-3689.) Additionally, for functional GPCR activity, the biological membrane spot may include necessary co-effectors and/or adaptors. Furthermore, biological membranes from lysated cells that contain a large number of cell surface molecules can be directly used to fabricate biomembrane arrays according to the present teachings.

In some embodiments of the present teachings, although the protein of one spot is different from that of another, the proteins can be related. In some embodiments, the two different proteins are members of the same protein family. The different proteins on the invention array may be either functionally related or just suspected of being functionally related. In some embodiments of the invention, however, the function of the immobilized proteins may be unknown. In this case, the different proteins on the different spots of the array share a similarity in structure or sequence or are simply suspected of sharing a similarity in structure or sequence. Alternatively, the proteins may be fragments of different members of a protein family. In a further embodiment of the invention, the proteins share similarity in pharmacological and physiological distribution or roles.

In some embodiments, the protein included in the spot differs from the protein included on a second spot of the same array. In such an embodiment, a plurality of different proteins are present on separate spots of the array. Typically the array comprises at least about two different proteins. In some embodiments of the array, each of the spots of the array comprises a different protein. For instance, an array comprising about 100 spots could comprise about 100 different proteins. Likewise, an array of about 10,000 spots could comprise about 10,000 different proteins. In some embodiments, however, each different protein is included on more than one separate spot on the array. For instance, each different protein may optionally be present on two to six different spots. An array of the invention, therefore, may comprise about five-thousand spots, but only comprise about one thousand different proteins since each different protein is present on three different spots.

In some embodiments, the array is fabricated using cell membrane preps. Such cell membrane preps contain a large number of different cell membrane proteins in addition to the membrane protein of interest. In some embodiments, cell membrane preps obtained from normal and diseased tissues can be used to form an array of the present teachings and the resulting array can be used to compare the pharmacological and physiological characteristics of the tissues.

In some embodiments, each of the spots of the array comprises the same protein of interest but in different amounts, and/or in different embedded environments. For example, the same receptor can be obtained from lysated cell membrane preps, or from purified receptor re-constituted in liposomes or micelles of different compositions. The resulting array can be used to examine the effect of the environment on the stability and functionality of the receptor. In a further alternative embodiment, each of the spots of the array comprises the same protein of interest but with different point mutations. The resulting arrays can be used to systematically examine the structure and function relationship of the receptor.

Uses of the Arrays

Biomembrane arrays according to the present teachings can be utilized in drug-membrane interactions. In addition to investigating protein-membrane binding, membrane arrays may be useful for screening drug-membrane interactions. These interactions can depend on the composition of the lipid membrane for example the content of cholesterol in the lipid bilayer. Cholesterol can further induce a change in the fluidity of the bilayer. The therapeutic and toxic effects of many drugs are affected by interactions with lipid membrane. To demonstrate the influence of the lipid composition on drug-membrane interactions, the fluidity changes introduced by a non-steroidal anti-inflammatory drug (NSAID), nimesulide, in bilayers with various contents of cholesterol. NSAIDs, (e.g. aspirin or ibuprofen) are the most important drugs for treatment of inflammation, pain, and fever were studied. The biomembranes stamped on arrays offer the attractive possibility of faithful reproduction of supported lipid bilayer biomembranes on substrates that can subsequently be used in automated high-throughput screening of predetermined drug targets. Furthermore, the arrays of the present teachings are particularly suited for use in drug development, medical diagnostics, biosensors, metabolomics and proteomics applications. Receptors and other membrane molecules known to be implicated in cellular processes can be screened against a panel of agonists or antagonists in high throughput, when such ligand-membrane molecule binding can be detected.

In some embodiments, arrays of biomembrane drug or protein association can be studied using a wide range of detection methods. As desired, detection can be either quantitative, semiquantitative, or qualitative. The arrays of the present teachings, can be interfaced with optical detection methods such as absorption in the visible or infrared range, chemiluminescence, and fluorescence (including lifetime, polarization, fluorescence correlation spectroscopy (FCS), and fluorescence-resonance energy transfer (FRET)). Furthermore, other modes of detection such as those based on optical waveguides (PCT Publication WO96/26432 and U.S. Pat. No. 5,677,196), surface plasmon resonance, surface charge sensors, surface force sensors, and MALDI-MS are compatible with many embodiments described herein.

The assays used on these arrays may be direct, noncompetitive assays or indirect, competitive assays. In the noncompetitive method, the affinity for binding sites on the probe is determined directly. In this method, the proteins in the spots are directly exposed to the analyte (“the target”). The analyte may be labeled or unlabeled. If the analyte is labeled, the methods of detection would include fluorescence, luminescence, radioactivity, etc. If the analyte is unlabeled, the detection of binding would be based on a change in some physical property at the probe surface. This physical property could be refractive index, or electrical impedance. The detection of binding of unlabeled targets could also be carried out by mass spectroscopy. In the competitive method, binding-site occupancy is determined indirectly. In this method, the proteins of the array are exposed to a solution containing a cognate labeled ligand for the probe array and an unlabelled target. The labeled cognate ligand and the unlabelled target compete for the binding sites on the probe protein spots.

In some embodiments of the present teachings, a method for screening a plurality of proteins for their ability to bind a particular component of a target sample is described. This method comprises delivering a test molecule, drug or protein sample to an array of the present teachings, and detecting, either directly or indirectly, for the presence or amount of the particular component retained at each spot. A test molecule can encompass all manner of organic, inorganic, biological and non-biological molecules that may be used in conjunction with the methods of the present teachings.

In some embodiments, the method further comprises the intermediate step of washing the array to remove any unbound or nonspecifically bound components of the sample from the array before the detection step. In some embodiments, the method further comprises the additional step of further characterizing the particular component retained on at least one spot.

In some embodiments of the invention, a method of assaying for drug-membrane protein and protein-membrane protein binding interactions is provided which comprises the following steps: first, delivering a sample comprising at least one protein or drug to be assayed for binding to the array of the present teachings; and then detecting, either directly, or indirectly, for the presence or amount of the protein or drug from the sample that is retained at each spot.

Some embodiments of the invention provide a method of assaying in parallel for the presence of a plurality of analytes in a sample which can react with one or more of the membrane proteins on the array. This method comprises delivering the sample to the array and detecting the interaction of the analyte with the membrane protein at each spot.

In some embodiments of the invention, a method of assaying in parallel for the presence of a plurality of analytes in a sample which can bind one or more of the proteins on the array comprises delivering the fluid sample to the array and detecting, either directly or indirectly, for the presence or amount of analyte retained at each spot. In some embodiments, the method further comprises the step of washing the array to remove any unbound or non-specifically bound components of the sample from the array.

The array may be used in a diagnostic manner when the plurality of analytes being assayed are indicative of a disease condition or the presence of a pathogen in an organism. In some embodiments, the sample which is delivered to the array can then typically be derived from a body fluid or a cellular extract from the organism.

The array may be used for drug screening when a potential drug candidate is screened directly for its ability to bind or otherwise interact with a plurality of proteins on the array. Alternatively, a plurality of potential drug candidates may be screened in parallel for their ability to bind or otherwise interact with one or more proteins on the array. The drug screening process may optionally involve assaying for the interaction, such as binding, of at least one analyte or component of a sample with one or more proteins on an array, both in the presence and absence of the potential drug candidate. This allows for the potential drug candidate to be tested for its ability to act as an inhibitor of the interaction or interactions originally being assayed.

Moreover, for GPCR arrays, it is preferable, in some embodiments, that the receptors immobilized are associated with one or more of their coeffectors such as G-proteins and G protein coupled receptor kinases (GRKs). In some embodiments, cell membrane preps from a cell line co-overexpressing a desired receptor and its coeffectors are used. In some embodiments, a reconstituted receptor in a liposome or micelle is used, in which the receptor is associated with one or more preferred coeffectors in a preferable ratio. The coupling of the receptor with its coeffectors can be carried out before or after the receptor is arrayed. The coeffectors can be either purified natural proteins, recombinant proteins with native sequences, or recombinant proteins with unique combinations of subunits such as mutants and chimeras.

Functional Assays in HERG Microarrays

In some embodiments membrane components can be used to incorporate into liposomes and then inked into hydrogel stamps. In some embodiments membrane components consisting of G-coupled receptors, ion channel proteins as well as those commercially sold by Perkin Elmer called Membrane Target Systems can be incorporated into liposomes to form proteoliposomes and stamped onto various substrates. Essentially, any membrane component can be utilized in the hydrogel stamps provided that they can be isolated and inserted into a lipid bilayer and remain functional. In some embodiments, cloned membrane receptors, cytokines, integrins, growth factors, ion channels can be cloned into a cell line such as Human Embryo Kidney 293 cells and expressed on the surface of the cell. Membrane preparations of cloned cells can then be isolated and incorporated into designed liposomes and used as inks in hydrogel stamps according to the present teachings. In some embodiments, HERG ion channel proteins, a class of potassium voltage gated Ion channels useful in the study of heart disease can be expressed recombinantly on eukaryotic cell membranes and purified. Alternatively, primary cells taken from animals, including humans expressing HERG ion cannels can be isolated, purified and the resulting cells can be manipulated (for example the membrane can be isolated with detergents and centrifuged) to yield functional HERG ion channel membrane components. The purified HERG membrane ion channels can then be added to various lipid bilayers of substantially identical lipid compositions or different lipid compositions to create proteoliposomes to be used in methods of the present teachings.

### EXAMPLES

Fabrication of Agarose Stamps

An aqueous solution containing 4% (w/v) of high-gel strength agarose (OmniPur; Merck, Darmstadt, Germany) in 0.15 M KCl to the boiling point and cast it onto a patterned PDMS master at room temperature. The molten agarose can be allowed to gel at room temperature and peeled off the PDMS master to obtain the agarose stamps. Depending on the desired dimensions of the agarose stamps, different PDMS masters can be employed to mold the stamps. The PDMS master for stamps with posts (raised zones) with 1 mm diameter was a replica (positive) of a PDMS replica (negative) of a standard 1536-well plate (polystyrene) with flat bottoms (Corning, Cambridge, Mass., USA). Masters can also be prepared by photolithography for stamps with posts with diameter of 200 and 700 μm. Depending on the PDMS master used for casting, arrays of posts on the agarose stamp, were (i) 200 μm in diameter, 130 μm in height, and spaced 200 μm from each other, (ii) 700 μm in diameter, 700 μm in height, and spaced 300 μm from each other; or, (iii) 1 mm in diameter, 1.5 mm in height, and spaced 1 mm from each other.

Preparation of Liposomes

Lipid mixtures used to prepare liposomes were: 99% L-α-phosphatidylcholine form chicken egg (egg PC; Sigma Aldrich) and 1% (w/w) 1,2-dipalmitoyl-sn-glycero-3-phosphoethanolamine-N-(lissamine-rhodamine B sulfonyl) (rh-PE; Avanti Polar Lipids); 97% egg PC and 3% NBD-labelled PE (NBD-PE; Avanti Polar Lipids); mixtures of egg PC/rh-PE or NBD-PE/1,2-dioleoyl-sn-glycero-3-[phospho-L-serine] (DOPS; Avanti Polar Lipids), and mixtures of egg PC/rh-PE/cholesterol (Avanti Polar Lipids). Small unilamellar liposomes can be produced by tip sonication using a Branson Sonifier 150 (Branson Ultrasonics Corporation, Danbury, USA) of 1 mg lipid in 500/L of an aqueous solution containing 0.15 M KCl, for 2-6 minutes (with ˜5 watts output energy). Before sonication, the lipids can be dissolved in chloroform and 100 μL of a 10 mg/mL lipid solution in chloroform can be used to deposit a lipid film on the wall of a 5 mL round bottom flask using a rotatory evaporator under vacuum (starting form −300 torr and going up to −740 torr). Residual traces of chloroform were removed by desiccation under vacuum (˜−740 torr) for at least 1 hour.

Cleaning of Microscope Glass Slides

Microscope glass slides (Microslides, No. 2974, Corning, N.Y.) can be cleaned with fresh piranha solution (mixture of concentrated sulfuric acid and 30% hydrogen peroxide) followed by washing with deionized water at least eight times and drying at 180° C. for 2 hours.

Inking and Stamping Procedure

To ink the agarose stamps with 1 mm or 700 μm posts, the stamps can be upside down in a Petri dish containing a solution of 0.15 M KCl, such that ¾ of the thickness of the stamp is immersed in the KCl solution and the posts (which were facing upwards) were out of the KCl solution. Posts can be inked in some embodiments individually by pipetting ˜0.2 μL of liposome suspension on top of each post (Figure S1b). Neighboring posts on the same stamp can be inked with different proteoliposome suspensions. Once the solution is adsorbed by the hydrogel (typically after ˜4 minutes), another droplet of ˜0.2 ˜L of solution can be added on top of each post and this process can be repeated for 4, 5 or more times. In case of stamps with smaller posts (200 μm in diameter), the agarose stamp can be inked by immersing the posts in a solution of liposomes for ˜30 min. After inking the stamps can be turned upside down (200 μm posts facing upwards) and after the stamp adsorbed all solution, the stamp is ready for stamping. In the beginning of a stamping series, the stamp can be stamped 4-7 times on clean glass slides to remove excess solution of proteoliposomes from the stamp's surface.

To form arrays of biomembranes, the inked agarose stamp is placed in contact with for example clean glass slides for 5-10 sec. After removing the stamp from the slides, the glass slides can be immediately immersed in water or PBS solution. In some embodiments, the stamping procedures can be carried out at room temperature with at least 50% humidity. In some embodiments carrying out the stamping procedure in humidity of less than 50% can result in supported bilayers with reduced fluidity. The stamped spots of lipid bilayers retained their fluidity even after storing them for two weeks in buffer solution.

Fluorescence Intensity After Multiple Stamping Without Re-Inking

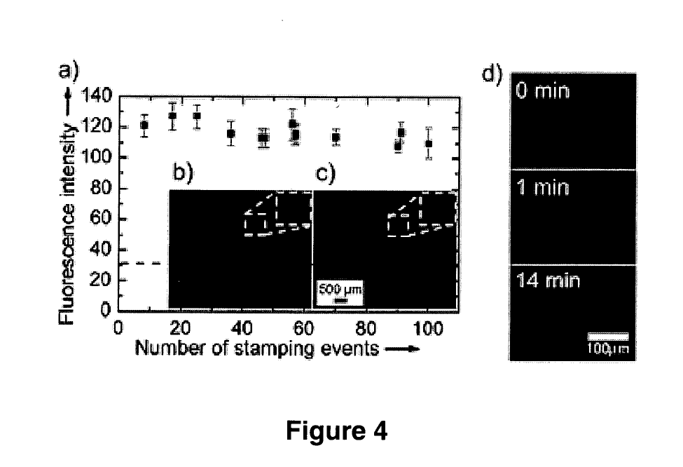

In some embodiments, the agarose stamp can be inked once, and then stamped in a pattern on any generally suitable substrate to produce 100 membrane arrays. In some embodiments more than 100 arrays can be stamped and in others less than 100 arrays would produce the desired results. The mean fluorescence intensity of the supported lipid bilayers can subsequently be measured. As shown in FIG. 4, after stamping the biomembranes described in the present teachings, the standard deviation of the fluorescence intensity within any individual spot can be less than 9.5% and from spot to spot in an array it can be less than 9%.

It will be apparent to those skilled in the art that various modifications and variations can be made to the present teachings without departing from the spirit and scope of the invention. Thus, it is intended that the present teachings cover the modifications and variations of this invention provided they come within the scope of the appended claims and their equivalents.

What is claimed is:

1. A process for making a biomembrane array comprising:
providing a first substrate having a discrete zone;
providing a second substrate;
loading a first ink having a proteoliposome onto said discrete zone to form a loaded zone; and
contacting said loaded zone to said second substrate such that said first ink is deposited from said loaded zone on said second substrate, thereby forming a biomembrane array on said second substrate.

2. The process according to claim 1, further comprising:
reloading said first ink onto said discrete zone to form a reloaded zone after said contacting said loaded zone to said second substrate such that said first ink is deposited from said loaded zone on said second; and
contacting said reloaded zone to said second substrate such that said first ink is deposited from said reloaded zone on said second substrate.

3. The process according to claim 1, wherein said loading said first ink having said proteoliposome comprises loading said first ink having at least one functional membrane protein.

4. The process according to claim 3, wherein said loading said first ink having at least one functional membrane protein comprises loading said first ink having a mammalian membrane protein.

5. The process according to claim 4, wherein said loading said first ink having said mammalian membrane protein comprises loading said first ink having a mammalian membrane protein isolated from mammalian cells.

6. The process according to claim 4, wherein said loading said first ink having said mammalian membrane protein comprises loading said first ink having a recombinant protein purified from a host cell.

7. The process according to claim 6, wherein said loading said first ink having a recombinant protein purified from a host cell comprises loading said first ink having said recombinant protein purified from a host cell selected from the group consisting of bacterial cells, yeast cells, plant cells, insect cells and animal cells.

8. The process according to claim 3, wherein said loading said first ink having said least one functional membrane protein comprises loading said first ink having at least one functional membrane protein having at least one of the group consisting of integral membrane proteins, transport proteins, receptors, enzymes, anchor proteins, heat shock proteins, trafficking proteins, cytokines, voltage and ligand gated ion channels.

9. The process according to claim 8, wherein said receptors include G-coupled protein receptors.

10. The process according to claim 8, wherein said voltage and ligand gated ion channels comprise voltage gated potassium ion channels.

11. The process according to claim 10, wherein said voltage gated potassium ion channels comprise voltage gated potassium ion channels HERG and Kv1.3.

12. The process according to claim 1, wherein said proteoliposome comprises liposomes having embedded membrane proteins.

13. The process according to claim 12, wherein said liposomes comprise at least one lipid selected from the group consisting of L-phosphatidylcholine, 1,2-dipalmitoyl-sn-glycero-3-phosphoethanolamine-N-(lissamine), phosphatidylethanloamine, phosphatidylserince, phosphatidic acid, phosphatidylinositol, phosphatidylglycerol, sphingomyelin, cholesterol and 1,2-dioleol-sn-glycero-3-[phospho-L-serine].

14. The process according to claim 1, wherein said providing a second substrate comprises providing a second substrate having a coated surface.

15. The process according to claim 14, wherein said providing a second substrate having a coated surface comprises providing a second substrate having a surface coated with one or more lipid compatible material that enhance the affinity of lipids to said second substrate.

16. The process according to claim 15, wherein said lipid compatible material is selected from the group consisting of streptavidin, positively charged amino groups, wheat germ agglutinin, collagen, lysine, bovine serum albumin and any natural or recombinant antibody which binds specifically to a bound membrane protein, polyvinylamines, polyallylamines, polyethyleneimines and modified polyethyleneimines.

17. The process according to claim 1, wherein said providing a second substrate comprises providing a second substrate being made of glass, metal, plastic, ceramic or silicon.

18. The process according to claim 1, wherein said providing a second substrate comprises providing a second substrate shaped as a slide, a chip, a wafer, a cell culture plate, or a Petri dish.

19. The process according to claim 1, wherein said providing a first substrate having a discrete zone comprises providing a first substrate having a discrete zone and being made at least in part of hydrogels selected from the group consisting of agarose, polyacrylamide, gelatin, alginate, chitosan, pluronic and collagen and combinations thereof.

20. The process according to claim 1, wherein said providing a first substrate having a discrete zone comprises providing a first substrate having agarose and being cast from a polydimethylsiloxane mold having raised zones.

21. A process for making a biomembrane array comprising:
providing a first substrate having a first discrete zone and a second discrete zone;
providing a second substrate;
loading a first ink having a proteoliposome onto said first discrete zone to form a first loaded zone and simultaneously loading a second ink having said proteoliposome onto a said second discrete zone to form a second loaded zone; and
contacting said first loaded zone and said second loaded zone to said second substrate such that said first ink is deposited from said first loaded zone to said second substrate and said second ink is deposited from said second loaded zone to said second substrate.

22. The process according to claim 21, wherein said first ink is different from said second ink.

23. The process according to claim 21, wherein said loading said first ink having said proteoliposome comprises loading said first ink having at least one functional membrane protein.

24. The process according to claim 23, wherein said loading said first ink having at least one functional membrane protein comprises loading said first ink having a mammalian membrane protein.

25. The process according to claim 24, wherein said loading said first ink having said mammalian membrane protein comprises loading said first ink having a mammalian membrane protein isolated from mammalian cells.

26. The process according to claim 24, wherein said loading said first ink having said mammalian membrane protein comprises loading said first ink having a recombinant protein purified from a host cell.

27. The process according to claim 26, wherein said loading said first ink having a recombinant protein purified from a host cell comprises loading said first ink having said recombinant protein purified from a host cell selected from the group consisting of bacterial cells, yeast cells, plant cells, insect cells and animal cells.

28. The process according to claim 23, wherein said loading said first ink having said least one functional membrane protein comprises loading said first ink having at least one functional membrane protein having at least one of the group consisting of integral membrane proteins, transport proteins, receptors, enzymes, anchor proteins, heat shock proteins, trafficking proteins, cytokines, voltage and ligand gated ion channels.

29. The process according to claim 28, wherein said receptors include G-coupled protein receptors.

30. The process according to claim 28, wherein said voltage and ligand gated ion channels comprise voltage gated potassium ion channels.

31. The process according to claim 30, wherein said voltage gated potassium ion channels comprise voltage gated potassium ion channels HERG and Kv1.3.

32. The process according to claim 21, wherein said proteoliposome comprises liposomes having embedded membrane proteins.

33. The process according to claim 32, wherein said liposomes comprise at least one lipid selected from the group consisting of L-phosphatidylcholine, 1,2-dipalmitoyl-sn-glycero-3-phosphoethanolamine-N-(lissamine), phosphatidylethanloamine, phosphatidylserince, phosphatidic acid, phosphatidylinositol, phosphatidylglycerol, sphingomyelin, cholesterol and 1,2-dioleol-sn-glycero-3-[phospho-L-serine].

34. The process according to claim 21, wherein said providing a second substrate comprises providing a second substrate having a coated surface.

35. The process according to claim 34, wherein said providing a second substrate having a coated surface comprises providing a second substrate having a surface coated with one or more lipid compatible material that enhance the affinity of lipids to said second substrate.

36. The process according to claim 35, wherein said lipid compatible material is selected from the group consisting of streptavidin, positively charged amino groups, wheat germ agglutinin, collagen, lysine, bovine serum albumin and any natural or recombinant antibody which binds specifically to a bound membrane protein, polyvinylamines, polyallylamines, polyethyleneimines and modified polyethyleneimines.

37. The process according to claim 21, wherein said providing a second substrate comprises providing a second substrate being made of glass, metal, plastic, ceramic or silicon.

38. The process according to claim 21, wherein said providing a second substrate comprises providing a second substrate shaped as a slide, a chip, a wafer, a cell culture plate, or a Petri dish.

39. The process according to claim 21, wherein said providing a first substrate having a first discrete zone comprises providing a first substrate having a first discrete zone and being made at least in part of hydrogels selected from the group consisting of agarose, polyacrylamide, gelatin, alginate, chitosan, pluronic and collagen and combinations thereof.

40. The process according to claim 21, wherein said providing a first substrate having a first discrete zone comprises providing a first substrate having agarose and being cast from a polydimethylsiloxane mold having raised zones.

41. A method of producing a hydrogel stamp comprising the steps:
casting a hydrogel polymer into a patterned manifold mold, the hydrogel polymer having one or more discrete zones, each zone having a surface;
loading at least one proteoliposome-containing ink onto said surface of said one or more discrete zones;
removing said casted patterned hydrogel stamp from the mold; and
applying at least one proteoliposome suspension ink onto said surface of each of said one or more discrete zones.

42. The method according to claim 41 wherein said applying at least one proteoliposome suspension ink onto said surface of each of said one or more discrete zones comprises applying a first proteoliposome suspension ink on to a first of said discrete zones and applying a second proteoliposome suspension ink on to a second of said discrete zones, said first proteoliposome suspension ink being different from said second proteoliposome suspension ink.

43. The method according to claim 41, wherein said casting a hydrogel polymer comprises casting a hydrogel polymer being made of a hydrogel selected from the group consisting of agarose, polyacrylamide, gelatin, alginate, chitosan, pluronic, collagen and combinations thereof.
